(19) United States
(12) Patent Application Publication
(10) Pub. No.: US 2021/0106248 A1
(43) Pub. Date: Apr. 15, 2021

(54) ECG Analysis System

(71) Applicant: DawnLight Technologies Inc., Palo Alto, CA (US)

(72) Inventors: Nan Du, Palo Alto, CA (US); Nan Liu, Palo Alto, CA (US); Jia Li, Palo Alto, CA (US)

(21) Appl. No.: 16/598,849

(22) Filed: Oct. 10, 2019

(51) Int. Cl. A61B 5/0452; G06T 11/60; G16H 50/20

(52) U.S. Cl. CPC A61B 5/0452; G06T 11/60; G06T 2207/30004; G06T 2207/20084; G16H 50/20

(57) ABSTRACT

A biometric signal graphical analysis system is described. In an embodiment, a graphical data preprocessing module is configured to receive a biometric graph and generate a normalized biometric graph. A graphical image analysis module is configured to receive and machine process the normalized biometric graph and generate a machine representation. A biometric information module generates an additional machine representation of biometric information combinable with the machine representation of the graphical image analysis module. A diagnosis module is configured to receive and combine the machine representation and the additional machine representation.

### FIELD OF THE INVENTION

The present disclosure relates generally to analysis of graphical biometric data including electrocardiograms (ECG). More specifically, the disclosure describes machine intelligence-based analysis and prediction based on graphical data.

### BACKGROUND

Graph-based biometric signals indicative of a condition of a human or animal can be obtained by the use of monitoring instrumentation. For example, electrocardiograms (ECG), electroencephalograms (EEG), photoplethysmographs (PPG), pneumograms, beat to beat blood pressure, and blood oxygen saturation instruments typically provide graphical output. These signals can be examined by medical doctors or experts to determine health of a patient and assist in providing a medical diagnosis. Such expert analysis is, however, time-consuming and expensive.

Machine intelligence-based signal analysis systems can provide improvements in speed, cost, and reproducibility of analysis. Unfortunately, graphical signals can be difficult to provide in digital form, and can be noisy, complex and highly variable, making automated analysis difficult. Fully or partially automated systems able to provide reliable diagnosis based at least in part on graphical, paper, or screen snapshots of biometric signals would be useful.

### SUMMARY

In one embodiment, a graphical data preprocessing module is configured to receive a biometric graph and generate a normalized biometric graph. A graphical image analysis module is configured to receive and machine process the normalized biometric graph and generate a machine representation. A biometric information module is configured to generate an additional machine representation of biometric information combinable with the machine representation of the graphical image analysis module. A diagnosis module is configured to receive and combine machine representations from the graphical image analysis module and the biometric information module.

Some embodiments also include:

The biometric graph being processed in near real-time.

The biometric graph being derived from at least one of paper, a photographic image, and video historical records.

The normalization further comprising at least one of text and annotation removal, conversion to gray scale, and pixel resizing.

The machine representation being derived at least in part from neural network processing.

The additional machine representation being derived at least in part from neural network processing.

The additional machine representation being derived at least in part from at least one of an electronic medical record, a patient profile, and genomic data.

The biometric graph being an electrocardiogram.

An implementation of another embodiment includes receiving a biometric graph by a graphical data preprocessing module. The graphical data preprocessing module generates a normalized biometric graph. A graphical image analysis module machine-processes the normalized biometric graph to generate a machine representation. A biometric information module generates an additional machine representation of biometric information, where the additional machine representation is combinable with the machine representation. A diagnosis module receives the machine representation and the additional machine representation, and combines the machine representation and the additional machine representation.

Some embodiments also include:

The biometric graph being processed in near real-time.

The biometric graph being derived from at least one of paper, a photographic image, and video historical records.

The normalization further comprising at least one of text and annotation removal, conversion to gray scale, and pixel resizing.

The machine representation being derived at least in part from neural network processing.

The additional machine representation being derived at least in part from neural network processing.

The additional machine representation being derived at least in part from at least one of an electronic medical record (EMR), a patient profile, and genomic data.

The biometric graph being an electrocardiogram.

Another embodiment includes a processing system receiving one or more ECG representations. The processing system performs a lead-based image reorganization on the ECG representations, and performs an image representation learning on the ECG representations. The processing system receives user data, and performs representation learning on the user data. The processing system combines results from the image representation learning and the representation learning to generate a joint model, and performs a prediction.

Some embodiments also include:

The ECG representations being associated with any combination of 6 ECG leads, 9 ECG leads, or 12 ECG leads.

The ECG representations being derived from at least of paper, a photographic image, and video historical records.

Any combination of the image representation learning and the representation learning being performed at least in part using neural network processing.

### BRIEF DESCRIPTION OF THE DRAWINGS

Non-limiting and non-exhaustive embodiments of the present disclosure are described with reference to the following figures, wherein like reference numerals refer to like parts throughout the various figures unless otherwise specified.

FIG. 1 is a block diagram depicting a system for automated graphical analysis and diagnosis.

FIG. 2 is a block diagram depicting an embodiment of a processing system used to implement certain functions of a biometric signal graphical analysis system.

FIG. 3 is a block diagram depicting an embodiment of a graphical image analysis module.

FIG. 4 is a block diagram depicting an embodiment of a biometric information module.

FIG. 5 is a flow diagram depicting an embodiment of a method to process a biometric graph.

FIG. 6 is a flow diagram depicting an embodiment of a method to generate a normalized biometric graph.

FIG. 7 is a flow diagram depicting an embodiment of a method to generate a prediction.

FIG. 8 is a flow diagram depicting an embodiment of another method to generate a prediction.

FIG. 9 is a schematic diagram illustrating a 12-lead ECG graph.

FIG. 10 is a schematic diagram illustrating a 9-lead ECG graph with blanked ECG leads omitted during preprocessing.

FIG. 11 is a block diagram depicting an embodiment of a system that includes a biometric signal graphical analysis system.

FIG. 12 is a schematic diagram that depicts a flow to generate a genome representation.

### DETAILED DESCRIPTION

The present disclosure relates in part to systems and methods that are configured to process graphical data associated with a user's biometric information. In some embodiments, a processing system is configured with machine learning algorithms that process this graphical data and generate a prediction. In particular embodiments, the machine learning algorithms are implemented using neural networks. The processing system is further configured to generate one or more predictions or diagnoses associated with a health condition of the user.

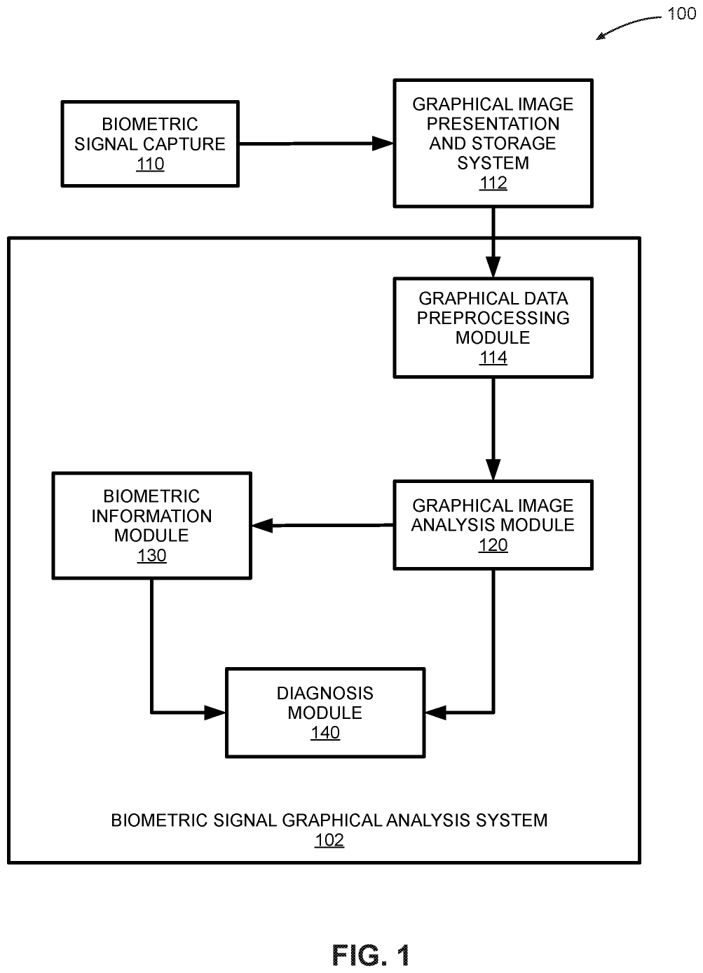

FIG. 1 is a block diagram depicting a system 100 for automated graphical analysis and diagnosis. In some embodiments, system 100 includes a biometric signal capture 110 that is configured to receive and capture (i.e., measure) one or more biometric signals associated with a user. Examples of biometric signals include electrocardiogram (ECG) signals, heartbeat signals, electroencephalogram (EEG) signals, and so on. The biometric signals captured by biometric signal capture 110 are received by a graphical image presentation and storage system 112 that is configured to store and display the captured biometric signals. For example, graphical image presentation and storage system may be comprised of any combination of hard drives, memory devices, visual display monitors, printers, audio speakers, physical file storage cabinets and associated electronic scanning facilities, and so on. In some embodiments, graphical image presentation and storage system 112 is configured to output stored biometric data to a biometric signal graphical analysis system 102, details of which are provided herein.

In some embodiments, biometric data stored by graphical image presentation and storage system 112 is received by a graphical data preprocessing module 114 that is included in biometric signal graphical analysis system 102. Graphical data preprocessing module 114 is configured to process the stored biometric data (also referred to herein as a “biometric graph”) and generate a normalized biometric graph using a normalization process. Details of the normalization process are described herein.

In some embodiments, the biometric graph is derived from any combination of paper records, photographic images, and video historical records. For example, a biometric graph may be an archived ECG computer file or a paper ECG file associated with a particular patient. Or, the biometric graph may be a digitized ECG file generated in substantially real-time. The normalized biometric graph is received by a graphical image analysis module 120 that is included in some embodiments of biometric signal graphical analysis system 102. In some embodiments, graphical image analysis module 120 is configured to machine process the normalized biometric graph and generate a machine representation of the normalized biometric graph. In particular embodiments, the machine processing is performed at least in part by a neural network or any other machine learning algorithm. Some embodiments use convolutional neural networks (CNNs) to implement machine processing, where such a CNN includes both convolutional and pooling components. The CNN processes the normalized biometric graph that may be presented to the CNN as a two-dimensional, three-dimensional, or higher-dimensional image, to generate a vector of a certain length. In some embodiments, this vector is referred to as an image representation, as described herein.

Some embodiments of biometric signal graphical analysis system 102 include a biometric information module 130 that is configured to receive biometric information from graphical image analysis module 120, and generate an additional machine representation of the biometric data. In some embodiments, the additional machine representation is derived at least in part from any combination of an electronic medical record, a patient profile, and genomic data that may be stored in a database. This additional machine representation is combinable with the machine representation generated by graphical image analysis module 120. In particular embodiments, the additional machine representation is generated using machine processing that is performed at least in part by a neural network or some other machine learning algorithm.

Outputs generated by graphical image analysis module 120 and biometric information module 130 (i.e., the machine representation and the additional machine representation, respectively) are received by a diagnosis module 140 that is included in some embodiments of biometric signal graphical analysis system 102. Diagnosis module 140 is configured to combine the machine representation and the additional machine representation and generate a prediction or a diagnosis of one or more health conditions associated with a user. In some embodiments, diagnosis module 140 is configured to analyze outputs from graphical image analysis module 120 to predict abnormalities in the outputs. In particular embodiments, outputs from graphical image analysis module 120 include processed ECG images, and diagnosis module 140 is configured to predict abnormalities such as arrhythmia, cardiac ischemia, and other health conditions. In some embodiments, diagnosis module 140 is configured to compare ECG curve features in the processed ECG images with previously learned features associated with one or more ECG image learning sets, to automatically specify what kind of arrhythmia or cardiac ischemia abnormalities exist in the ECG.

In some embodiments, biometric signal graphical analysis system 102 performs processing on the biometric graph and biometric information in real-time or near real-time. Embodiments of biometric signal graphical analysis system 102 can be implemented on processing systems such as a laptop computer, a desktop computer, a server, a cloud server, a field-programmable gate array (FPGA), a digital signal processor (DSP), or any other processing system. In other words, all processing (including machine processing) performed by graphical data preprocessing module 114, graphical image analysis module 120, biometric information module 130, and diagnosis module 140 is performed in real-time or near real-time. Essentially, the functionality of biometric signal graphical analysis system 102 parallels that of a human who is trained to read and interpret medical records such as an ECG. In the case of biometric signal graphical analysis system 102, associated image recognition, artificial intelligence and machine learning algorithms are trained to read and interpret medical records (e.g., a biometric graph such as an ECG), and provide a diagnosis of a health condition in a manner that a trained human would. Some embodiments of biometric signal graphical analysis system 102 may be used as a diagnosis tool to alert a medical professional (e.g., a doctor, a physician assistant or a nurse) about a health condition or an abnormality that might have been overlooked by the medical professional.

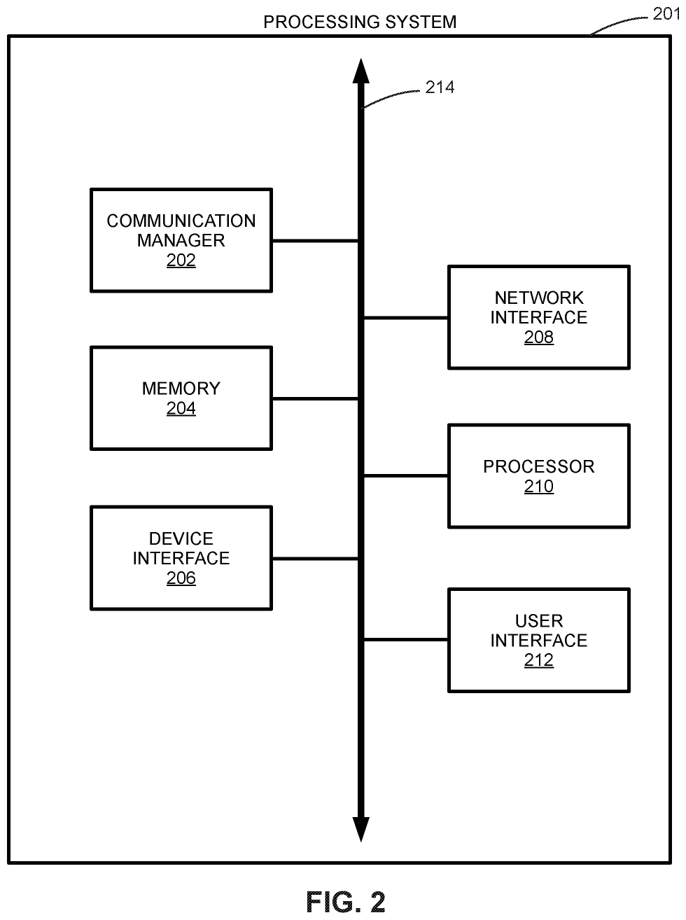

FIG. 2 is a block diagram depicting an embodiment of a processing system 201 used to implement certain functions of biometric signal graphical analysis system 102. In some embodiments, processing system 201 includes a communication manager 202, where communication manager 202 is configured to manage communication protocols and associated communication with external peripheral devices as well as communication within other components in processing system 201. For example, communication manager 202 may implement and manage communication protocols between graphical data preprocessing module 114 and graphical image presentation and storage system 112. Communication manager 202 may also be responsible for managing communication between the different components within processing system 201.

Some embodiments of processing system 201 include a memory 204 that may include both short-term memory and long-term memory. Memory 204 may be used to store, for example, temporary and permanent data associated with a biometric graph. Memory 204 may be comprised of any combination of hard disk drives, flash memory, random access memory, read-only memory, solid state drives, and other memory components.

In some embodiments, processing system 201 includes a device interface 206 that is configured to interface processing system 201 with one or more external devices such as an external hard drive, an end user computing device (e.g., a laptop computer or a desktop computer), and so on. Device interface 206 generates any necessary hardware communication protocols associated with one or more communication protocols such as a serial peripheral interface (SPI), a serial interface, a parallel interface, a USB interface, and so on.

A network interface 208 included in some embodiments of processing system 201 includes any combination of components that enable wired and wireless networking to be implemented. Network interface 208 may include an Ethernet interface, a Wi-Fi interface, and so on. In some embodiments, network interface 208 allows biometric signal graphical analysis system 102 to send and receive data over a local network or a public network.

Processing system 201 also includes a processor 210 configured to perform functions that may include generalized processing functions, arithmetic functions, and so on. Processing system 201 is configured to process, for example, one or more biometric graphs. Any artificial intelligence algorithms or machine learning algorithms (e.g., neural networks) associated with biometric signal graphical analysis system 102 may be implemented using processor 210.

In some embodiments, processing system 201 may include a user interface 212, where user interface 212 may be configured to receive commands from a user (such as a medical professional, a health care worker, family member or friend of a patient, etc.), or display information to the user. User interface 212 enables a user to interact with biometric graphical analysis system 102. In some embodiments, user interface 212 includes a display device to output data to a user; one or more input devices such as a keyboard, a mouse, a touchscreen, one or more push buttons, and one or more switches; and other output devices such as buzzers, loudspeakers, alarms, LED lamps, and so on.

A data bus 214 included in some embodiments of processing system 201 is configured to communicatively couple the components associated with processing system 201 as described above.

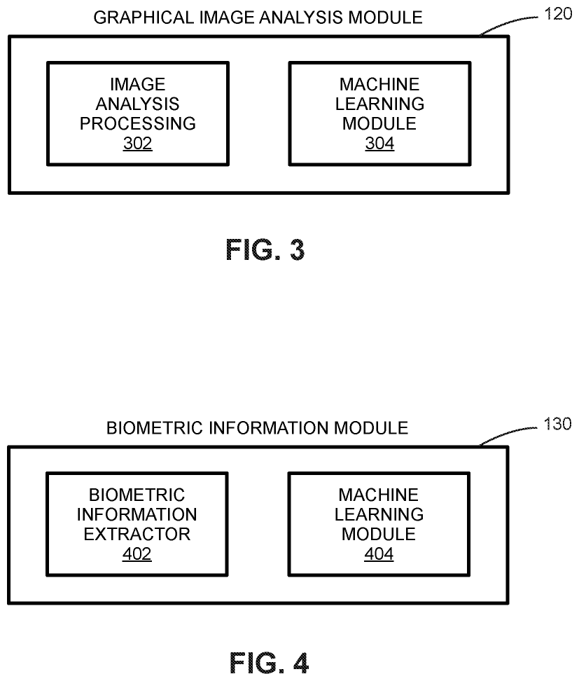

FIG. 3 is a block diagram depicting an embodiment of graphical image analysis module 120. In some embodiments, graphical image analysis module 120 includes an image analysis processing 302 that is configured to implement image processing algorithms and other algorithms that are configured to process a biometric graph. Graphical image analysis module 120 also includes a machine learning module 304 that is configured to process a biometric graph using one or more machine learning algorithms. In some embodiments, machine learning module 304 may include one or more neural networks.

FIG. 4 is a block diagram depicting an embodiment of biometric information module 130. In some embodiments, biometric information module 130 includes a biometric information extractor 402 that is configured to perform computing that enables biometric information module 130 to extract biometric information from any combination of an electronic medical record, a patient profile, and genomic data that may be stored in a database. Biometric information module 130 also includes a machine learning module 404 that is configured to extract biometric information from any combination of an electronic medical record, a patient profile, and genomic data that may be stored in a database, using one or more machine learning algorithms. In some embodiments, machine learning module 404 may include one or more neural networks.

In some embodiments, graphical image analysis module 120 is configured as a representation learning module for the biometric graph, which extracts informative information from a raw image (i.e., the biometric graph) into a vector. In some embodiments, biometric information module 130 is a representation learning module configured to process additional medical information (e.g., genome data, an EMR, and so on). Learned representations from graphical image analysis module 120 and biometric information module 130 are processed together by diagnosis module 140 that itself is configured to learn a model to predict any abnormalities in ECG data.

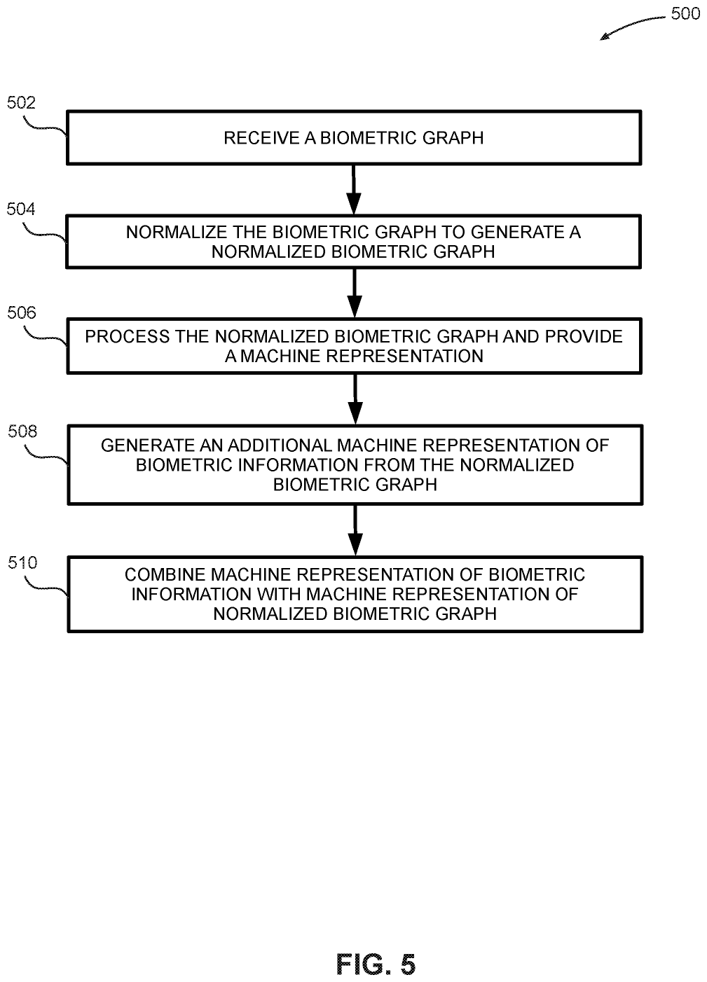

FIG. 5 is a flow diagram depicting an embodiment of a method 500 to process a biometric graph. At 502, the method receives a biometric graph. This step may be related to graphical data preprocessing module 114 receiving data from graphical image presentation and storage system 112. Next, at 504, the method normalizes the biometric graph to generate a normalized biometric graph, as accomplished by graphical data preprocessing module 114. At 506, the method processes the normalized biometric graph and provides (i.e., generates) a machine representation. This step is accomplished by graphical image analysis module 120. Next, at 508, the method generates an additional machine representation of biometric information from the normalized biometric graph. This step is accomplished by biometric information module 130, as described herein. Finally, at 510, the method combines the machine representation of the biometric information with the machine representation of the normalized biometric graph to generate a prediction (i.e., the machine representation and the additional machine representation), as performed by diagnosis module 140.

Some embodiments used to implement method 500 involve processing the biometric graph in real-time or near-real-time. The biometric graph may be derived from any combination of paper records, one or more photographic images, and video historical records associated with a user or a patient. In some embodiments, the normalization includes any combination of text and annotation removal, conversion to gray scale, and pixel resizing. In particular embodiments, each of the machine representation and the additional machine representation are independently generated at least in part from neural network processing. In some embodiments, the additional machine representation is derived at least in part from at least one of an electronic medical record, a patient profile, and genomic data. In particular embodiments, the biometric graph is an ECG.

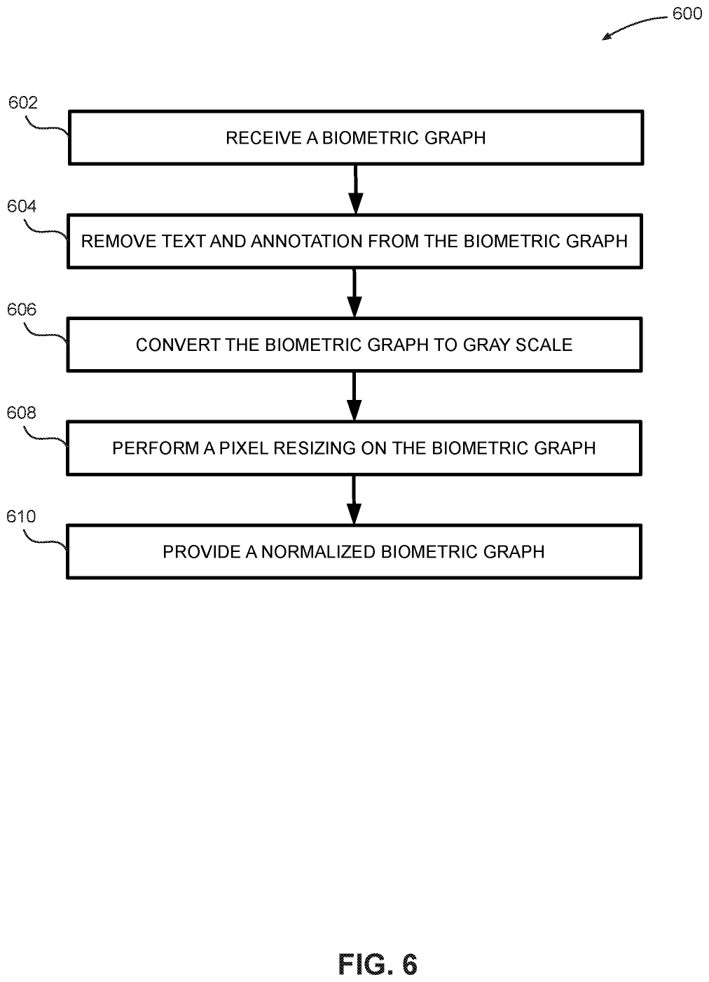

FIG. 6 is a flow diagram depicting an embodiment of a method 600 to generate a normalized biometric graph. Method 600 presents an example of the steps that may be used to implement step 504 of method 500. At 602, the method receives a biometric graph. At 604, the method removes text and annotation from the biometric graph. For example, if the biometric graph is a digital file generated by scanning a paper ECG, the biometric graph may include notes written up by a medical professional who may have reviewed the paper ECG in the past. Such notes are removed from the digital file associated with the biometric graph in step 604.

At 606, the method converts the biometric graph to gray scale. Next, at 608, the method performs a pixel resizing on the biometric graph. In some embodiments, the pixel resizing converts a biometric graph (i.e., an image representation) into an image of predetermined dimensions. For example, the biometric graph may be of 2160×1900 in pixel dimensions. The pixel resizing converts this biometric graph to an image of 600×400 in pixel dimensions. In particular embodiments, 600×400 in pixel dimensions is considered to be a standard (i.e., a predetermined) format. Finally, at 610, the method provides (generates) a normalized biometric graph. In some embodiments, generating a normalized biometric graph reorganizes multiple ECG leads in a formal order. This allows method 500 to process ECG data with a lower number of leads. For example, a standard training data model associated with method 500 may be associated with 12 ECG leads. To apply this data to a 9-lead case, a 12-lead image is downgraded to a 9-lead image in a specific format, then the standard training data model is applied to the 9-lead image. Other embodiments of method 600 may implement steps 604 through 608 in an order different from the order presented herein.

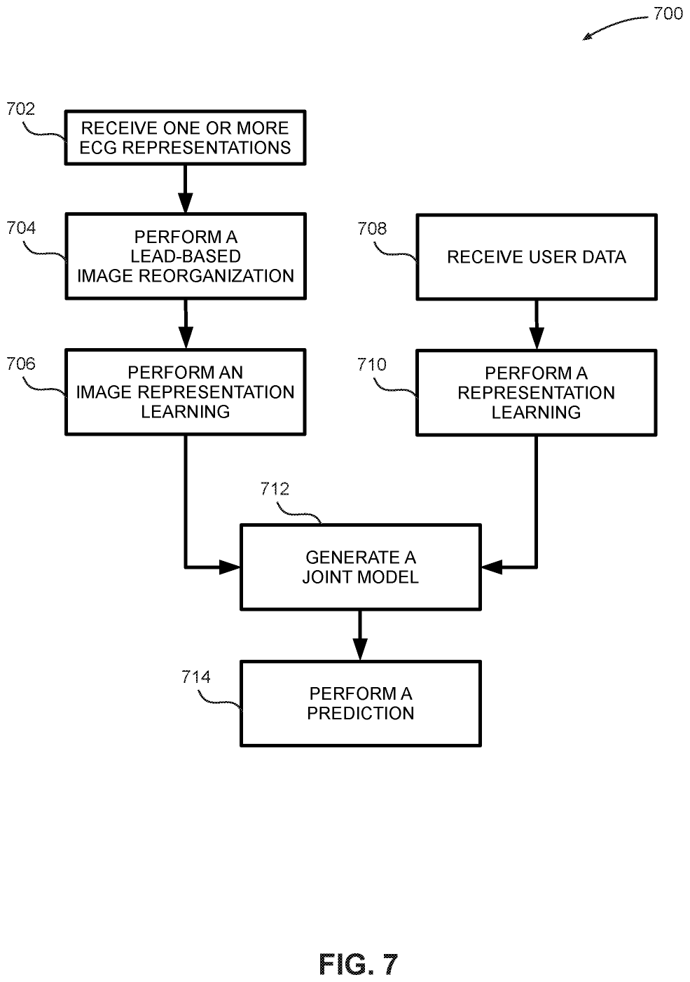

FIG. 7 is a flow diagram depicting an embodiment of a method 700 to generate a prediction. Method 700 is configured to process one or more ECG representations, which are a special class of biometric graphs. At 702, the method receives one or more ECG representations. In some embodiments, the ECG representations may be digitized files based on archived paper ECGs. In other embodiments, the ECG representations may be comprised of real-time digital data. This step is a special case of biometric signal graphical analysis system receiving a biometric graph. Next, at 704, the method performs a lead-based image reorganization. This step is implemented to account for a variability in a number of leads used to generate an ECG; ECGs may be generated using, for example, 6, 9, or 12 leads. Step 704 performs a task of associating a specific ECG trace with a specific set of leads. Next, at 706, the method performs an image representation learning. In some embodiments, this task is performed by machine learning algorithms. In particular embodiments, the machine learning algorithms are realized using one or more neural networks. For example, the neural network may be a CNN-based network with both convolutional and pooling components. The neural network transforms any image such as a biometric graph in two dimensions, three dimensions, or higher dimensions, into a certain length vector that used as an output vector.

In a parallel process, at 708, the method 700 receives user data. In some embodiments, the user data is any combination of data from an electronic medical record, a patient profile, and genomic data associated with the user (or patient). At 710, the method performs a representation learning using, for example, machine learning algorithms. In particular embodiments, the machine learning algorithms are realized using one or more neural networks. For example, text data in an EMR is processed using a bag of words, word embedding or graph embedding, which converts the text data into a final vector representation.

In some embodiments, steps 702 through 706 correspond to functionalities of graphical data preprocessing module 114 and graphical image analysis module 120, while steps 708 and 710 corresponds to a functionality of biometric information module 130. At the next step, 714, the results from step 706 and step 708 are combined to generate a joint model. In some embodiments, this combination is performed by diagnosis module 140, which is also configured to perform a prediction at step 714. In some embodiments, this prediction is associated with diagnosis module predicting or diagnosing a health condition associated with a user or a patient.

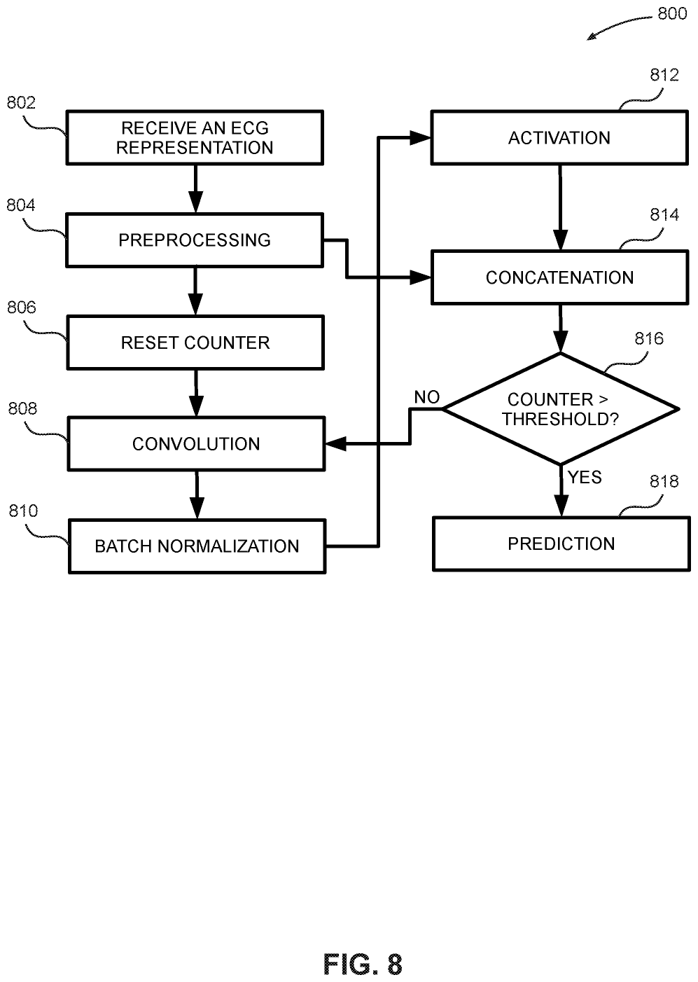

FIG. 8 is a flow diagram depicting an embodiment of another method 800 to generate a prediction. At step 802, the method receives an ECG representation. In some embodiments, this ECG representation may be any combination of paper records, a photographic image, and video historical records. At 804, the method performs preprocessing on the ECG representation. For example, this step may be performed by graphical data preprocessing module 114. At 806, the method resets a counter used to count a number of iterations, or loops, performed by method 800. Next, at 808, the method performs a convolution on the ECG representation using, for example, one or more convolutional neural networks to convert a multi-dimensional image into a vector representation, as described earlier. Next, at 810, the method performs a batch normalization, while at 812 the method performs an activation. At 812, the method performs a concatenation. In some embodiments, the batch normalization is performed to normalize the data to prevent a gradient function associated with the data from vanishing. An activation process is used to activate the neural network via a nonlinear function such as sigmoid, RELU, PRELU, and so on. The concatenation process links two vectors tail to head together. At 816, the method checks to determine whether the counter is greater than a specific threshold (e.g., 16 iterations). If the counter is less than the threshold, then the method returns to 808, and the cycle repeats. If, at 816, the counter is greater than the threshold, then the method goes to 818, where the method performs a prediction, such as determining a health condition associated with a patient or a user.

In some embodiments, a highway associated with a deep learning architecture may be used to proceed directly from step 804 to step 814. This highway attempts to improve an overall performance of method 800 by linking a representation from a lower layer to a higher layer in the processing algorithm.

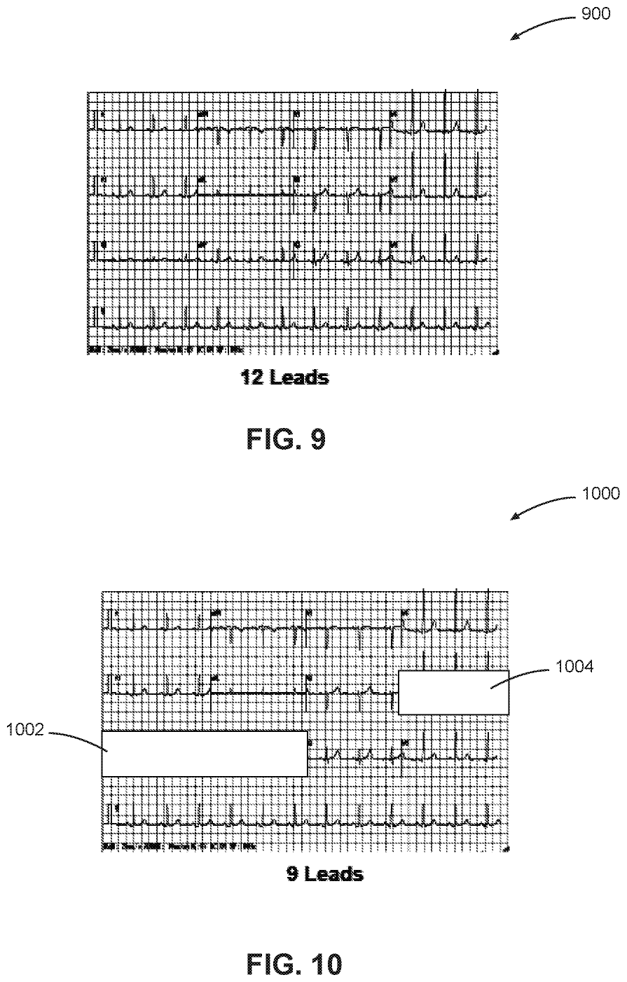

FIG. 9 is a schematic diagram illustrating a 12-lead ECG graph 900. 12-lead ECG graph 900 depicts an example of a biometric graph that may be received by biometric signal graphical analysis system 102. In some embodiments, ECG data associated with 12-lead ECG graph 900 may be a digitized file generated from a paper ECG. In other embodiments, ECG data associated with 12-lead ECG graph 900 may be generated from ECG data captured in real-time. 12-lead ECG graph 900 is processed by biometric signal graphical analysis system 102 to generate a prediction, using the systems and methods described herein.

FIG. 10 is a schematic diagram illustrating a 9-lead ECG graph 1000 with blanked ECG leads omitted during preprocessing. Specifically, FIG. 10 depicts a blanked-out region 1002 and a blanked-out region 1004 that together depict blanked out (i.e., omitted) ECG leads. 9-lead ECG graph 1000 depicts an example of a biometric graph that may be received by biometric signal graphical analysis system 102. In some embodiments, ECG data associated with 9-lead ECG graph 1000 may be a digitized file generated from a paper ECG. In other embodiments, ECG data associated with 9-lead ECG graph 1000 may be generated from ECG data captured in real-time. 9-lead ECG graph 1000 is processed by biometric signal graphical analysis system 102 to generate a prediction, using the systems and methods described herein. For example, step 704 may be used to perform a lead-based image reorganization, to account for any ECG leads omitted during preprocessing (i.e., blanked out region 1002 and blanked out region 1004). While FIG. 10 depicts 9-lead ECG graph 1000, other numeric configurations of ECG leads are possible (e.g., 6 ECG leads). Biometric signal graphical analysis system 102 is configured to process ECG data associated with any arbitrary numeric configurations of ECG leads.

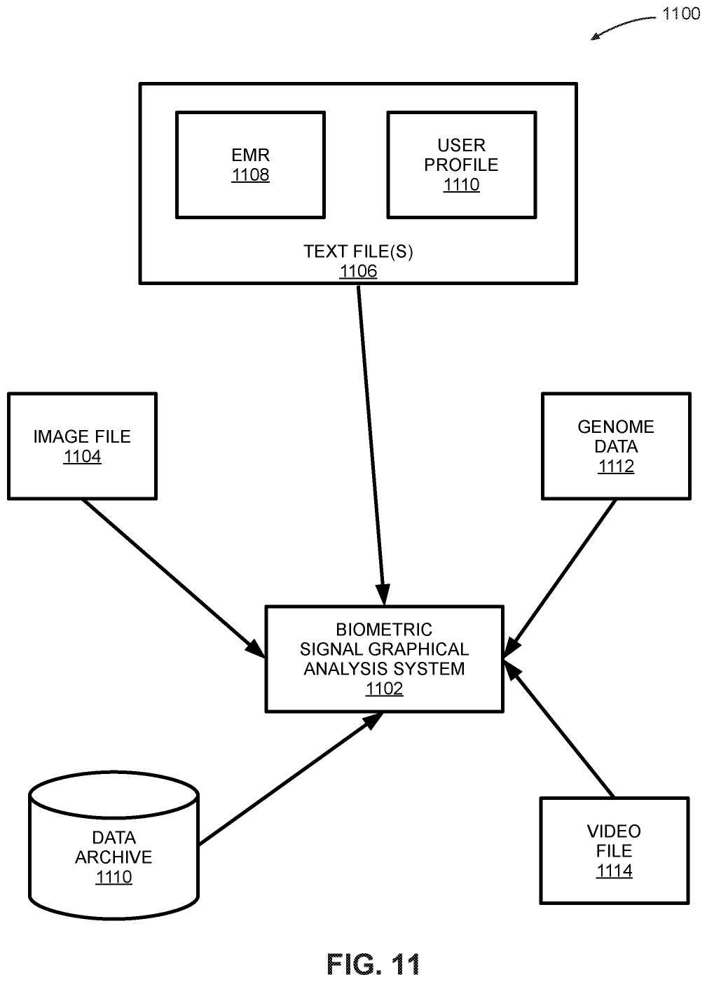

FIG. 11 is a block diagram depicting an embodiment of a system 1100 that includes a biometric signal graphical analysis system 1102. As shown in FIG. 11, system 1100 is comprised of biometric signal graphical analysis system 1102 reading biometric graphs from disparate sources. In some embodiments, biometric signal graphical analysis system 1102 reads one or more biometric graphs from a text file(s) 1106 that further includes an EMR 1108 that includes one or more electronic medical records associated with a patient or a user, and a user profile 1110 associated with the user. In some embodiments, EMR 1108 and user profile 1110 are text files that contain textual data.

Some embodiments of system 1100 include biometric signal graphical analysis system 1102 reading an image file 1104. In particular embodiments, image file 1104 may include one or more image files in any combination of JPEG, TIFF, PNG, or other image formats. In some embodiments, biometric signal graphical analysis system 1102 reads in a biometric graph from a data archive 1110. Data archive 1110 is a database that is configured to store archived medical records associated with the user. These archived medical records are read in as biometric graphs by biometric signal graphical analysis system 1102.

In some embodiments, biometric signal graphical analysis system 1102 reads in data from a genome data 1112, where genome data 1112 is configured to generate and store genome data associated with a user. In particular embodiments, this genome data is used by biometric information module 130. For example, biometric signal graphical analysis system 1102 checks to determine whether there are some certain annotations in the genome data (i.e., biomarkers or tags), which can be used as features to predict a final diagnosis. A process used to generate genome data is described herein. In some embodiments, biometric signal graphical analysis system 1102 reads in a video file 1114, where video file 1114 includes one or more video files associated with the patient. In a clinical domain, video may be recorded for one or tests associated with a patient. For example, a recognition test involves a patient answering a series of questions. A video is taken to record the patient's words and facial reactions.

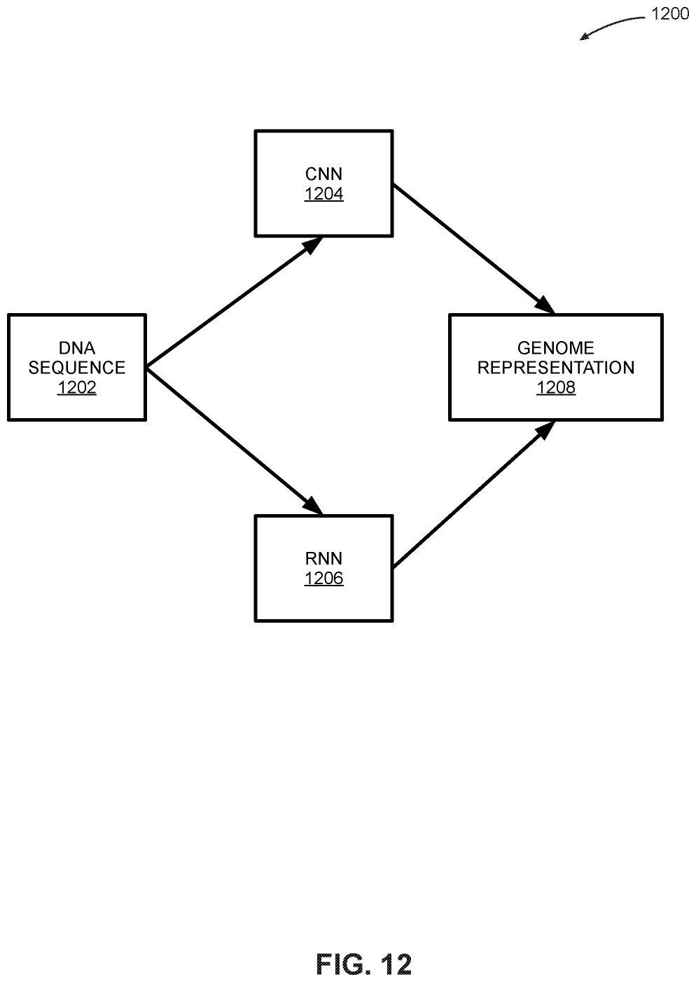

FIG. 12 is a schematic diagram that depicts a flow 1200 to generate a genome representation. As shown in FIG. 12, flow 1200 uses a DNA sequence 1202 as a basis for generating the genome representation. In some embodiments, data associated with the DNA sequence is curated, and then processed by a CNN 1204, and an RNN 1206, where CNN 1204 is a convolutional neural network, and RNN 1206 is a recursive neural network. A combined output from CNN 1204 and RNN 1206 is used to generate a genome representation 1208.

In some embodiments, CNN 1204 is configured to scan through genome data associated with DNA sequence 1202 while considering the genome data as a one-dimensional image. The genome data analyzed by CNN 1204 may include five nucleobases—adenine (A), cytosine (C), guanine (G), thymine (T), and uracil (U). These five nucleobases, called primary or canonical nucleobases, function as the fundamental units of genetic code and are the basic units of DNA composition. In some embodiments, CNN 1204 and RNN 1206 translate the genome data into a one-dimensional sequence (vector). In particular embodiments, CNN 1204 assigns a predefined number for each nuclease associated with the genome data. For instance, 0 for A, 1 for C, and so on so forth. Thus, ‘ACTG’ can be represented as ‘0123,’ which can be read by a 1-dimensional CNN. For RNN 1206, besides a predefined representation, a representation of the ACTG can be learned through RNN 1206 via a way of word embedding. This is accomplished by iteratively sequentially inputting nuclease data associated with DNA sequence 1202, for instance, A->C->T->G. In this way, CNN 1204 and RNN 1206 attempt to learn an embedding (i.e., a ‘representation’) for the nuclease. After the entire genome sequence associated with DNA sequence 1202 is analyzed, CNN 1204 and RNN 1206 learn an overall representation of DNA sequence 1202, which can be further used for generating genome representation 1208, and a final prediction.

In the foregoing description, reference is made to the accompanying drawings that form a part thereof, and in which is shown by way of illustration specific exemplary embodiments in which the disclosure may be practiced. These embodiments are described in sufficient detail to enable those skilled in the art to practice the concepts disclosed herein, and it is to be understood that modifications to the various disclosed embodiments may be made, and other embodiments may be utilized, without departing from the scope of the present disclosure. The foregoing detailed description is, therefore, not to be taken in a limiting sense.

Reference throughout this specification to “one embodiment,” “an embodiment,” “one example,” or “an example” means that a particular feature, structure, or characteristic described in connection with the embodiment or example is included in at least one embodiment of the present disclosure. Thus, appearances of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

Embodiments in accordance with the present disclosure may be embodied as an apparatus, method, or computer program product. Accordingly, the present disclosure may take the form of an entirely hardware-comprised embodiment, an entirely software-comprised embodiment (including firmware, resident software, micro-code, etc.), or an embodiment combining software and hardware aspects that may all generally be referred to herein as a “circuit,” “module,” or “system.” Furthermore, embodiments of the present disclosure may take the form of a computer program product embodied in any tangible medium of expression having computer-usable program code embodied in the medium.

Any combination of one or more computer-usable or computer-readable media may be utilized. For example, a computer-readable medium may include one or more of a portable computer diskette, a hard disk, a random-access memory (RAM) device, a read-only memory (ROM) device, an erasable programmable read-only memory (EPROM or Flash memory) device, a portable compact disc read-only memory (CDROM), an optical storage device, a magnetic storage device, and any other storage medium now known or hereafter discovered. Computer program code for carrying out operations of the present disclosure may be written in any combination of one or more programming languages. Such code may be compiled from source code to computer-readable assembly language or machine code suitable for the device or computer on which the code will be executed.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction and then scaled accordingly. A cloud model can be composed of various characteristics (e.g., on-demand self-service, broad network access, resource pooling, rapid elasticity, and measured service), service models (e.g., Software as a Service (“SaaS”), Platform as a Service (“PaaS”), and Infrastructure as a Service (“IaaS”)), and deployment models (e.g., private cloud, community cloud, public cloud, and hybrid cloud).

The flow diagrams and block diagrams in the attached figures illustrate the architecture, functionality, and operation of possible implementations of systems, methods, and computer program products according to various embodiments of the present disclosure. In this regard, each block in the flow diagrams and/or block diagrams may represent a module, segment, or portion of code, which includes one or more executable instructions for implementing the specified logical function(s). It will also be noted that each block of the block diagrams and/or flow diagrams, and combinations of blocks in the block diagrams and/or flow diagrams, may be implemented by special purpose hardware-based systems that perform the specified functions or acts, or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flow diagram and/or block diagram block or blocks.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims. It is also understood that other embodiments of this invention may be practiced in the absence of an element/step not specifically disclosed herein.

1. A biometric signal graphical analysis system, comprising
a graphical data preprocessing module configured to receive a biometric graph and generate a normalized biometric graph;
a graphical image analysis module configured to receive and machine process the normalized biometric graph and generate a machine representation;
a biometric information module generating an additional machine representation of biometric information combinable with the machine representation of the graphical image analysis module; and
a diagnosis module configured to receive and combine the machine representation and the additional machine representation.

2. The biometric signal graphical analysis system of claim 1, wherein the biometric graph is processed in near real-time.

3. The biometric signal graphical analysis system of claim 1, wherein the biometric graph is derived from at least one of paper, a photographic image, and video historical records.

4. The biometric signal graphical analysis system of claim 1, wherein the normalization further comprises at least one of text and annotation removal, conversion to gray scale, and pixel resizing.

5. The biometric signal graphical analysis system of claim 1, wherein the machine representation is derived at least in part from neural network processing.

6. The biometric signal graphical analysis system of claim 1, wherein the additional machine representation is derived at least in part from neural network processing.

7. The biometric signal graphical analysis system of claim 1, wherein the additional machine representation is derived at least in part from at least one of an electronic medical record, a patient profile, and genomic data.

8. The biometric signal graphical analysis system of claim 1, wherein the biometric graph is an electrocardiogram.

9. A method for biometric signal graphical analysis, comprising:
receiving, by a graphical data preprocessing module, a biometric graph;
generating, by the graphical data preprocessing module, a normalized biometric graph;
machine processing, by a graphical image analysis module, the normalized biometric graph to generate a machine representation;
generating, by a biometric information module, an additional machine representation of biometric information, wherein the additional machine representation is combinable with the machine representation;
receiving, by a diagnosis module, the machine representation and the additional machine representation; and
combining, by the diagnosis module, the machine representation and the additional machine representation.

10. The method of claim 9, wherein the biometric graph is processed in near real-time.

11. The method of claim 9, wherein the biometric graph is derived from at least one of paper, a photographic image, and video historical records.

12. The method of claim 9, wherein the normalization further comprises at least one of text and annotation removal, conversion to gray scale, and pixel resizing.

13. The method of claim 9, wherein the machine representation is derived at least in part from neural network processing.

14. The method of claim 9, wherein the additional machine representation is derived at least in part from neural network processing.

15. The method of claim 9, wherein the additional machine representation is derived at least in part from at least one of an electronic medical record, a patient profile, and genomic data.

16. The method of claim 9, wherein the biometric graph is an electrocardiogram.

17. A method for processing ECG representations, comprising:
receiving, by a processing system, one or more ECG representations;
performing, by the processing system, lead-based image reorganization on the ECG representations;
performing, by the processing system, an image representation learning on the ECG representations;
receiving, by the processing system, user data;
performing, by the processing system, representation learning on the user data;
combining, by the processing system, results from the image representation learning and the representation learning to generate a joint model; and
performing, by the processing system, a prediction.

18. The method of claim 17, wherein the ECG representations are associated with any combination of 6 ECG leads, 9 ECG leads, or 12 ECG leads.

19. The method of claim 17, wherein the ECG representations are derived from at least one of paper, a photographic image, and video historical records.

20. The method of claim 17, wherein any one of the image representation learning and the representation learning is performed at least in part using neural network processing.
